(19) United States
(12) Patent Application Publication
(10) Pub. No.: US 2012/0202820 A1
(43) Pub. Date: Aug. 9, 2012

(54) PHARMACEUTICAL COMPOSITION HAVING THE ACTIVE SUBSTANCES METFORMIN AND SITAGLIPTIN OR VILDAGLIPTIN

(75) Inventors: Katrin Rimkus, Muenchen (DE); Ralph Stefan, Ebenweiler (DE)

(73) Assignee: Ratiopharm GMBH, Ulm (DE)

(21) Appl. No.: 13/496,346

(22) PCT Filed: Sep. 13, 2010
(86) PCT No.: PCT/EP10/63383

§ 371 Date: Mar. 29, 2012

(30) Foreign Application Priority Data
Sep. 15, 2009 (EP) 09170260.5

(51) Int. Cl. A61K 31/4985; A61P 3/10; A61K 31/40

(52) U.S. Cl. 514/249; 514/423

(57) ABSTRACT

The present invention relates to a pharmaceutical composition that comprises the active substance metformin in combination with one of the active substances sitagliptin or vildagliptin. The present invention further relates to a method of production of said pharmaceutical composition.

The present invention relates to a pharmaceutical composition that comprises the active substance metformin in combination with one of the active substances sitagliptin or vildagliptin. Furthermore, the present invention relates to a method of production of said pharmaceutical composition.

Metformin is a drug from the biguanides group, which is used in non-insulin-dependent diabetes (type 2 diabetes mellitus) and in particular for excess weight and obesity. Metformin is one of the antidiabetics longest in use. Metformin is the 1,1-dimethylbiguanide with the following structural formula:

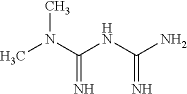

Metformin is available in strengths of 500 mg, 850 mg and 1000 mg, to allow adjustment to an individual blood sugar level. The tablets are administered orally. Metformin is used first as monotherapy. If this does not produce a sufficient lowering of blood sugar, it is known to combine the active substance with other oral antidiabetics, such as the dipeptidyl-peptidase-4 inhibitors.

Known dipeptidyl-peptidase-4 inhibitors are for example sitagliptin and vildagliptin. Sitagliptin is (R)-3-amino-1-[3-(trifluoromethyl)-5,6,7,8-tetrahydro[1,2,4]triazole[4,3-a]pyrazin-7-yl]-4-(2,3,5-trifluorophenyl)butan-1-one, which has the following structural formula:

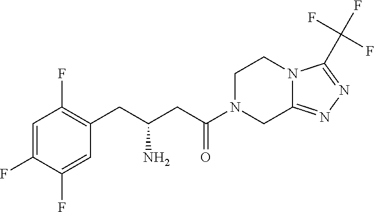

Vildagliptin is (2S)-{((3-hydroxyadamantan-1-yl)amino)acetyl}pyrrolidine-2-carbonitrile with the following structural formula:

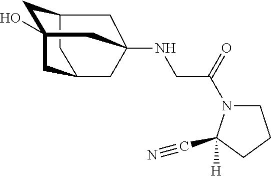

Sitagliptin is obtainable under the trade names Januvia® and Xelevia®. Combination preparations of sitagliptin and metformin are obtainable under the trade names Janumet® and Velmecia®. Vildagliptin is obtainable as a medicinal product under the trade name Galvus®, and a combination preparation of vildagliptin and metformin is obtainable under the trade name Eucreas®.

Combination preparations of metformin and vildagliptin are described in WO 2007/041053. The tablets disclosed can contain, in addition to the active substances, usual excipients, for example fillers, binders, disintegrants, lubricants and colorants. Examples of lubricants that are mentioned are colloidal silica, magnesium trisilicate, starch, talc, calcium phosphate, magnesium stearate, aluminium stearate, calcium stearate, magnesium carbonate, magnesium oxide, polyethylene glycol, cellulose and microcrystalline cellulose. It is said to be possible for the lubricant to be present in an amount of up to 6 wt. %. In the examples, the tablets are produced by methods of wet granulation.

WO 2007/078726 discloses combination preparations that contain 3 to 20 wt. % of dipeptidyl-peptidase-4 inhibitor, 25 to 94 wt. % of metformin hydrochloride, 0.1 to 10 wt. % of lubricant and 0 to 35 wt. % of binder. The lubricants mentioned are magnesium stearates, calcium stearates, stearic acid, sodium stearyl fumarate and hydrogenated castor oil. The tablets, which are produced in the examples by methods of wet granulation, preferably contain only up to 2 wt. % of lubricant.

The combination preparations described in the prior art have the drawback that the tablets can only be produced by wet granulation, as the active substance metformin is a very poorly compressible active substance. In wet granulation there is, however, the risk that the active substance will either be decomposed through interactions with the solvent used, and undesirable degradation products will be formed.

There is therefore still a need for pharmaceutical compositions that contain the active substance metformin in combination with one of the active substances sitagliptin or vildagliptin, and that can be produced by a simple method, preferably by direct compression. Moreover, it is desirable for the tablets obtained to have a hardness of >100 kN, suitable for varnishing. At the same time, addition of further excipients should not cause the tablets to become so large that they are difficult to swallow. Finally, the excipients must be selected so as to ensure rapid release of the active substances from the tablet.

Surprisingly, these problems were solved according to the invention by processing the active substances into the pharmaceutical composition together with more than 10 wt. % of lubricant, wherein the lubricant is polyethylene glycol or a mixture of polyethylene glycol with one or a plurality of other lubricants. The use of high lubricant concentrations in the pharmaceutical composition is all the more surprising, as it is known that the advantage of their lubricating action is often opposed by the disadvantage of hydrophobisation of the product and therefore a lengthening of the disintegration time or of the dissolution rate of the tablet, so that lubricants should be used in the lowest possible concentration (Schmidt Christin, Wirk- and Hilfsstoffe (Active substances and excipients), Wissenschaftliche Verlagsgesellschaft mbH, Stuttgart, 1999). Contrary to these known disadvantages of lubricants, it was found in the present work that if the lubricant is or comprises polyethylene glycol, it can be used in high concentration of more than 10 wt. % and nevertheless tablets with a high dissolution rate are obtained, which can moreover be produced by direct compression and which have a hardness that is for example advantageous for varnishing.

The present invention therefore relates to a pharmaceutical composition that comprises the active substance metformin or a pharmaceutically compatible salt thereof in combination with one of the active substances sitagliptin or vildagliptin or a pharmaceutically compatible salt of one of these active substances and more than 10 wt. % of lubricant relative to the total weight of the composition, wherein the lubricant is polyethylene glycol or a mixture of polyethylene glycol with one or a plurality of other lubricants.

The pharmaceutical composition according to the invention is a composition with a fixed dose of the active substances, wherein both active substances are contained together in a unit dose, in particular a tablet.

The active substance metformin is used in the pharmaceutical composition according to the invention preferably as pharmaceutically compatible salt and in particular as hydrochloride salt.

The active substance sitagliptin is preferably used in the form of one of its pharmaceutically compatible salts. Pharmaceutically compatible salts of sitagliptin are described for example in WO 2003/004498. Particularly preferably, sitagliptin is used as its phosphate salt, in particular as phosphate monohydrate. The corresponding salt and its production are disclosed in WO 2005/003135. Alternatively, the active substance sitagliptin can be used as hydrochloride, sulphate, mesylate, besylate, tosylate or mono-, di- or tricarboxylic acid salt. Suitable carboxylic acids have the structure R¹—COON, in which R¹ is hydrogen, carboxyl, C₁₋₄-alkyl or C₂₋₄-alkenyl and the alkyl or alkenyl group can be substituted with 1-2 carboxyl, 1-3 hydroxyl, 1-3 amino, 1-3 phenyl and/or 1-3 C₁₋₅-alkyl residues. Preferred carboxylic acids are fumaric acid, malonic acid, malic acid, succinic acid, lactic acid, glycolic acid, maleic acid, citric acid, aspartic acid and mandelic acid.

The active substance vildagliptin can be used in the form of its free base or, if desired, in the form of a pharmaceutically compatible salt thereof. Pharmaceutically compatible salts of vildagliptin are disclosed in WO 2000/034241.

The amounts of the active substances in the pharmaceutical composition according to the invention can be freely selected by a person skilled in the art depending on the desired dosage. Preferably the pharmaceutical composition contains 25 to less than 87 wt. % of metformin hydrochloride, 3 to 20 wt. % of sitagliptin or vildagliptin or a pharmaceutically compatible salt of one of these active substances, calculated on the basis of the free base of the active substance, and more than 10 to 30 wt. % of lubricant, in each case relative to the total weight of the composition. It is to be noted that in the present text, the figures for percentage by weight, if they relate to the total weight of the composition, are relative to the weight of the composition, but without any tablet coatings in the form of varnish layers, etc. that may be present.

The amounts of the active substances are preferably selected so that a unit dose of the pharmaceutical composition contains 50 mg or 100 mg of sitagliptin or vildagliptin, in each case calculated on the basis of the free base of the active substance, and 500 mg, 850 mg or 1000 mg of metformin hydrochloride. A particularly preferred tablet contains 50 mg of sitagliptin or vildagliptin, calculated on the basis of the free base of the active substance, and 1000 mg of metformin hydrochloride.

The pharmaceutical composition according to the invention contains, in addition to the active substances, as necessary further constituent, more than 10 wt. % of lubricant relative to the total weight of the composition. The lubricant is either polyethylene glycol or a mixture of polyethylene glycol with one or a plurality of other lubricants. The pharmaceutical composition according to the invention preferably contains 12 to 28 wt. %, preferably more than 15 to 28 wt. %, for example 15.1 to 24 wt. %, particularly preferably 16 to 24 wt. %, for example about 19 wt. % of lubricant, in each case relative to the total weight of the composition.

The polyethylene glycol used preferably has a molecular weight of at least 1000 g/mol. The molecular weight of the polyethylene glycol is preferably in the range from 1000 to 20000 g/mol, particularly preferably in the range from 6000 to 10000 g/mol. A preferred polyethylene glycol is PEG 8000.

If the lubricant is a mixture of polyethylene glycol and one or a plurality of other lubricants, the polyethylene glycol can be mixed with conventional known lubricants, for example magnesium stearate, calcium stearate, stearic acid, sodium stearyl fumarate, hydrogenated castor oil, talc, fumaric acid, starches, for example pea, wheat, maize, potato, rye, rice, algal or tapioca starch, sodium lauryl sulphate, colloidal silica, magnesium trisilicate, calcium phosphate, aluminium stearate, magnesium carbonate, magnesium oxide, cellulose and microcrystalline cellulose. Preferably the polyethylene glycol is mixed with one or a plurality of other lubricants, selected from the group consisting of talc, starch and sodium lauryl sulphate.

The mixture ratio of the lubricants used can be freely selected by a person skilled in the art depending on the desired properties of the pharmaceutical composition. The lubricant mixture should preferably contain at least 10 wt. %, preferably at least 50 wt. % and particularly preferably at least 85 wt. % of polyethylene glycol relative to the total weight of the lubricants.

In order to influence the release properties of the pharmaceutical composition, sodium lauryl sulphate can be used as lubricant addition. In this case the amount of sodium lauryl sulphate used is preferably 0.5 to 2 wt. % relative to the total weight of the composition.

In addition to the active substances and the lubricant, the pharmaceutical composition according to the invention can also contain further usual excipients, for example antioxidants, binders, emulsifiers, colorants, fillers and disintegrants. However, in a preferred embodiment the pharmaceutical composition does not contain any further ingredients, apart from the two active substances (wherein the active substance metformin hydrochloride can be mixed with Aerosil (colloidal silica)) and the lubricant. Particularly preferably the pharmaceutical composition consists of the two active substances, Aerosil and polyethylene glycol. Alternatively the pharmaceutical composition can consist of the two active substances, optionally Aerosil, polyethylene glycol and a binder, for example polyvinylpyrrolidone (PVP; povidone).

The pharmaceutical composition according to the invention can be in the form of tablets. These can preferably be obtained by direct compression or methods containing wet granulation or fusion granulation. Preferably the tablets are obtained by direct compression.

If desired, the tablets can be provided with one or a plurality of coatings, for example a film coating. Corresponding coatings are known by a person skilled in the art.

Finally, the present invention also relates to a method of production of a pharmaceutical composition as described above, wherein the active substances are mixed with the lubricant and optionally further excipients and the resultant mixture is compressed to tablets, optionally after sieving and/or granulation. Preferably the mixture is not granulated prior to compression, but compressed to tablets directly. Alternatively the mixture can first be formed into granules by wet or dry granulation or fusion granulation and then compressed to tablets.

In a preferred embodiment of the method according to the invention, the water content of the mixture is adjusted prior to compression to 2 to 3 wt. % relative to the total weight of the mixture. This improves the properties of the mixture, in particular for direct compression. The water content can be adjusted before or after sieving the mixture.

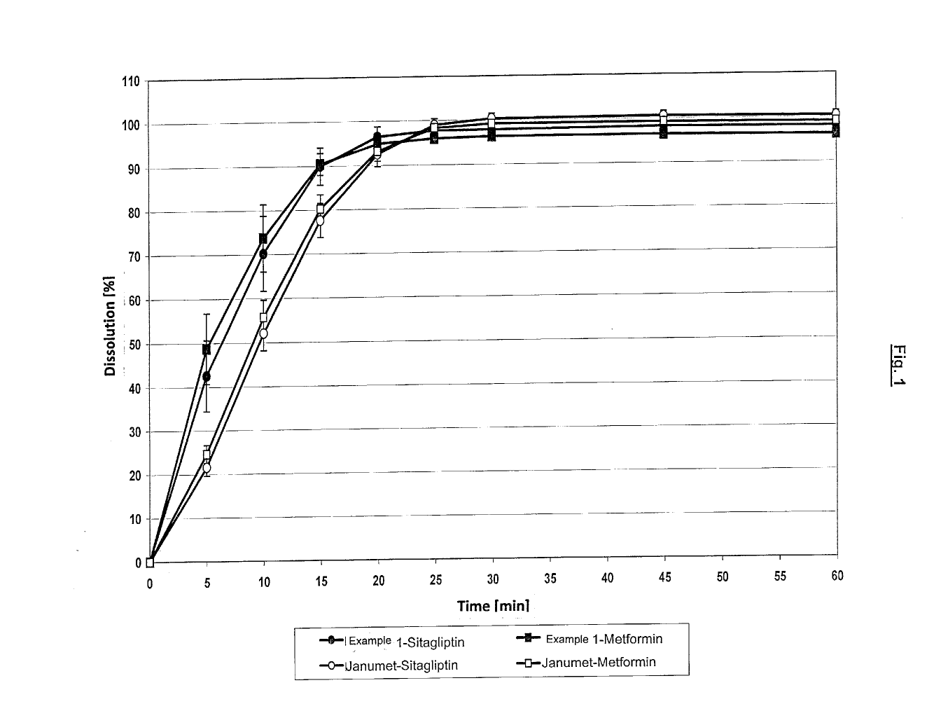

The accompanying FIG. 1 shows the release profiles of a pharmaceutical composition according to the invention according to example 1 for the two active substances sitagliptin and metformin in comparison with the commercial preparation Janumet®.

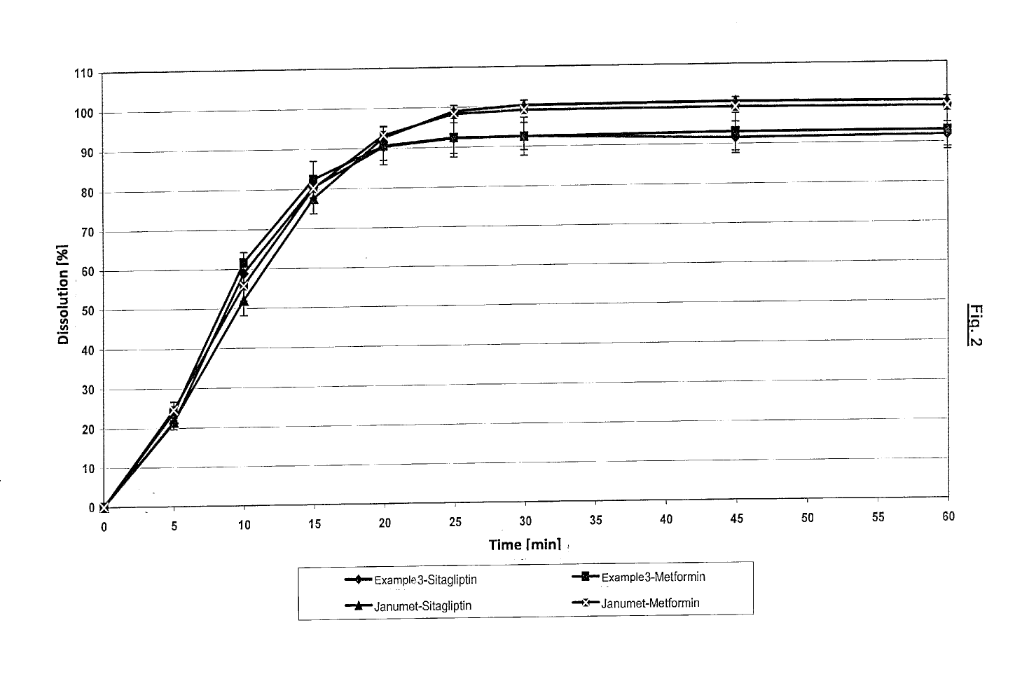

FIG. 2 shows the release profiles of a pharmaceutical composition according to the invention according to example 3 for the two active substances sitagliptin and metformin in comparison with the commercial preparation Janumet®.

The invention will now be explained in more detail by means of the following examples, which are not to be construed as limiting.

### EXAMPLE 1

| | | | Initial weight |
|---|---|---|---|
| Active substances and excipients | [mg/tablet] | [%] | [g/preparation] |
| Sitagliptin phosphate monohydrate | 63.13 | 4.79 | 1.26 |
| (79.2%) | | | |
| Metformin hydrochloride (99.6%) | 1004.02 | 76.23 | 20.08 |
| Aerosil 0.5% | | | |
| PEG 8000 | 250.00 | 18.98 | 5.00 |

The metformin hydrochloride, as mixture with 0.5% Aerosil, was mixed with sitagliptin phosphate monohydrate and PEG for 15 minutes in a tumbling mixer at 23 rpm in the Turbula T10B. The mixture was sieved on a 0.6 mm sieve and then compressed on an eccentric press. The tablet size was 21×11 mm.

The tablets were then coated in a drum coater (Lödige LHC 25) with 0.35 wt. % of Opadry II (15 wt. % in water).

The dissolution profile of the tablets obtained was measured for the active substances sitagliptin and metformin using 900 ml of phosphate buffer, pH 6, at 37° C. and 75 rpm by the paddle method (USP App. II). The dissolution profiles for the two active substances are shown in FIG. 1, wherein the dissolution profiles for the two active substances sitagliptin and metformin are shown together with the commercial product Janumet® for comparison. It can be seen that the tablets according to the invention release the active substances even more quickly than the commercial product.

### EXAMPLE 2

| | | | Initial weight |
|---|---|---|---|
| Active substances and excipients | [mg/tablet] | [%] | [g/preparation] |
| Vildagliptin | 50.45 | 3.87 | 1.01 |
| Metformin hydrochloride (99.6%) | 1004.02 | 76.97 | 20.08 |
| Aerosil 0.5% | | | |
| PEG 8000 | 250.00 | 19.16 | 5.00 |

Production of the tablets was carried out as in example 1.

### EXAMPLE 3

| | | | Initial weight |
|---|---|---|---|
| Active substances and excipients | [mg/tablet] | [%] | [g/preparation] |
| Sitagliptin phosphate monohydrate | 63.13 | 4.79 | 9.47 |
| (79.2%) | | | |
| Metformin hydrochloride (99.6%) | 1004.02 | 76.23 | 150.60 |
| Aerosil 0.5% | | | |
| PEG 8000 | 250.00 | 15.18 | 30.00 |
| PVP | 50.00 | 3.80 | 7.50 |

The mixture of active substances and excipients was melted and processed to granules. The granules were compressed to tablets as in example 1.

The tablets were then coated in a drum coater (Lödige LHC 25) with 0.35 wt. % of Opadry II (15 wt. % in water).

The dissolution profiles of the tablets obtained were determined as in example 1 and are presented in FIG. 2.

1. A pharmaceutical composition comprising, in combination:
a. a first active substance comprising metformin or a pharmaceutically compatible salt thereof;
b. a second active substance comprising sitagliptin or a pharmaceutically compatible salt thereof or (ii) vildagliptin or a pharmaceutically compatible salt thereof; and
c. a lubricant comprising more than 10 wt % relative to the total weight of the composition, wherein the lubricant is polyethylene glycol or a mixture, of polyethylene glycol with one or more other lubricants.

2. The pharmaceutical composition according to claim 1, wherein the first active substance comprises a hydrochloride salt of metformin.

3. The pharmaceutical composition according to claim 1, wherein the second active substance comprises a phosphate salt of sitagliptin.

4. The pharmaceutical composition according to claim 1, wherein said composition further comprises: (a) 25 to less than 87 wt. % metformin hydrochloride as the first active substance, (b) 3 to 20 wt. % of the second active substance, and (c) more than 10 to 30 wt. % lubricant, wherein active substance wt % is calculated on the basis of the free base of the active substance, further wherein wt % in each case is relative to the total weight of the composition.

5. The pharmaceutical composition according to claim 4, wherein said composition further comprises 12 to 28 wt. % lubricant relative to the total weight of the composition.

6. The pharmaceutical composition according to claim 1, wherein the lubricant comprises a polyethylene glycol having a molecular weight of at least 1000 g/mol.

7. The pharmaceutical composition according to claim 6, wherein the lubricant comprises a polyethylene glycol having a molecular weight ranging from 1000 to 20000 g/mol.

8. The pharmaceutical composition according to claim 1, wherein the lubricant comprises a mixture of polyethylene glycol with one or more other lubricants selected from the group consisting of talc, starch and sodium lauryl sulphate.

9. The pharmaceutical composition according to claim 8, wherein the mixture contains at least 10 wt. % polyethylene glycol relative to the total weight of the lubricant.

10. The pharmaceutical composition according to claim 8, wherein the mixture contains 0.5 to 2 wt. % sodium lauryl sulphate relative to the total weight of the composition.

11. The pharmaceutical composition according to claim 1, wherein said composition is in the form of a tablet.

12. A method of producing a pharmaceutical composition according to claim 1, wherein the first and second active substances are combined with the lubricant, alone or in combination with optional further excipients, to form a mixture and the resultant mixture is compressed to tablets.

13. The method according to claim 12, wherein the mixture further comprises water, further wherein the water content in the mixture is adjusted to 2 to 3 wt. % relative to the total weight of the mixture.

14. The pharmaceutical composition according to claim 3, wherein the phosphate salt comprises a phosphate monohydrate.

15. The pharmaceutical composition according to claim 5, wherein said composition further comprises 15 to 28 wt. % lubricant, relative to the total weight of the composition.

16. The pharmaceutical composition according to claim 7, wherein the lubricant comprises a polyethylene glycol having a molecular weight ranging from 6000 to 10000 g/mol.

17. The pharmaceutical composition according to claim 9, wherein the mixture contains at 50 wt. % polyethylene glycol relative to the total weight of the lubricant.

18. The pharmaceutical composition according to claim 11, wherein said tablet is obtained by direct compression or wet granulation.

19. The method of producing a pharmaceutical composition according to claim 12, wherein the resultant mixture is sieved and/or granulated prior to compression.
